(19) United States
(12) Patent Application Publication
(10) Pub. No.: US 2018/0220927 A1
(43) Pub. Date: Aug. 9, 2018

(54) ELECTROMAGNETIC NAVIGATION SYSTEM WITH MAGNETO-RESISTIVE SENSORS AND APPLICATION-SPECIFIC INTEGRATED CIRCUITS

(71) Applicant: Boston Scientific Scimed Inc., Maple Grove, MN (US)

(72) Inventors: David W. Kelly, Eagan, MN (US); Mathew Hein, Eden Prairie, MN (US)

(21) Appl. No.: 15/888,569

(22) Filed: Feb. 5, 2018

Related U.S. Application Data
(60) Provisional application No. 62/455,299, filed on Feb. 6, 2017.

(51) Int. Cl. A61B 5/06; G01D 5/16

(52) U.S. Cl. CPC A61B 5/062; A61B 2562/0223; G01D 5/16

(57) ABSTRACT

A sensing apparatus includes a magneto-resistive sensing element and a coil element formed on a first chip, and semiconductor circuitry formed on a second chip. The magneto-resistive sensing element senses magnetic fields, while the coil element is used to reset a magnetic orientation of the magneto-resistive sensing element. The semiconductor circuitry includes a reset circuit that controls the coil element and an amplifier circuit coupled to the magneto-resistive sensing element. The amplifier circuit operates to generate a sensing signal that is proportional to the sensed magnetic fields. The sensing signal is then used to activate the reset circuit.

### CROSS-REFERENCE TO RELATED APPLICATION

This application claims priority to Provisional Application No. 62/455,299, filed Feb. 6, 2017, which is herein incorporated by reference in its entirety.

### TECHNICAL FIELD

The present disclosure relates to systems, methods, and devices for tracking items. More specifically, the disclosure relates to systems, methods, and devices for electro-magnetically tracking medical devices used in medical procedures.

### BACKGROUND

A variety of systems, methods, and devices can be used to track medical devices. Tracking systems can use externally generated magnetic fields that are sensed by at least one tracking sensor in the tracked medical device. The externally generated magnetic fields provide a fixed frame of reference, and the tracking sensor senses the magnetic fields to determine the location and orientation of the sensor in relation to the fixed frame of reference.

### SUMMARY

In Example 1, a sensing apparatus comprising a magneto-resistive (MR) sensing element for sensing magnetic fields, a reset coil element configured to generate a reset field for resetting a magnetic orientation of the MR sensing element, and semiconductor circuitry including a reset circuit configured to supply a reset current to the reset coil element, and an amplifier circuit coupled to the MR sensing element. The amplifier circuit includes an amplifier output and is configured to generate a sensing signal proportional to the sensed magnetic fields. The MR sensing element and the coil element are formed on a first chip and the semiconductor circuitry is formed on a second chip.

In Example 2, the sensing apparatus according to Example 1, wherein the semiconductor circuitry is further configured to generate a reset control signal operative to activate the reset circuit and cause the reset circuit to supply the reset current to the reset coil element.

In Example 3, the sensing apparatus according to Example 2, wherein the amplifier circuit is configured to generate the reset control signal upon detecting a triggering event.

In Example 4, the sensing apparatus of Example 3, wherein the triggering event includes the amplifier output being short-circuited.

In Example 5, the sensing apparatus of any of Examples 1-4, further comprising a gain setting resistor, wherein the amplifier circuit is further coupled to the gain setting resistor; and wherein the sensing signal is based on a gain determined from a ratio of a resistance value of gain setting resistor to a resistance value of the MR sensing element.

In Example 6, the sensing apparatus of Example 5, wherein the gain setting resistor is formed on the first chip.

In Example 7, the sensing apparatus of any of Examples 1-6, wherein the semiconductor circuitry further includes a bias signal compensation circuit, wherein the amplifier circuit is further coupled to the bias signal compensation circuit, and wherein the bias signal compensation circuit generates a compensation signal based on the sensing signal, the compensation signal to be combined with a bias signal to the MR sensing element.

In Example 8 the sensing apparatus of Example 7, further comprising a tuning resistor formed on the first chip, wherein the compensation signal is based on a resistance value of the tuning resistor.

In Example 9, the sensing apparatus of Example 7, wherein the bias signal compensation circuit increases the compensation signal when the bias signal to the MR sensing element decreases, and decreases the compensation signal when the bias signal to the MR sensing element increases.

In Example 10, the sensing apparatus of any of Examples 1-9, wherein the first chip is placed in close proximity to the second chip.

In Example 11, the sensing apparatus of any of Examples 1-10, wherein the first chip and the second chip are electrically connected to one another.

In Example 12, the sensing apparatus of any of Examples 1-11, wherein the first chip is placed on top of the second chip.

In Example 13, a sensor assembly comprising a plurality of sensing apparatuses according to any of Examples 1-12 mechanically coupled to a substrate.

In Example 14, the sensor assembly of Example 13, wherein the substrate is a flexible substrate.

In Example 15, the sensor assembly of either of Examples 13 or 14, wherein the substrate includes a first portion oriented in a first plane and a second portion oriented in a second plane that is non-parallel to the first plane.

In Example 16, the sensor assembly of Example 15, wherein the second plane is oriented orthogonally to the first plane.

In Example 17, the sensor assembly of either of Examples 15 or 16, wherein a first one of the plurality of sensing apparatuses is supported by the first portion of the substrate, and a second one of the plurality of sensing apparatuses is supported by the second portion of the substrate.

In Example 18, a medical probe including a distal portion having a sensor assembly according to any of Examples 13-17.

In Example 19, a medical system comprising the medical probe according to Example 18, a magnetic field generator configured to generate a multi-dimensional magnetic field in a volume including the medical probe and a patient, and a processor operable to receive outputs from the sensor assembly to determine a position of the sensor assembly within the volume.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 shows a schematic of a tracking system, in accordance with certain embodiments of the present disclosure.

FIG. 2 shows a schematic of sensor circuitry, in accordance with certain embodiments of the present disclosure.

FIG. 3 shows a schematic of sensor circuitry, in accordance with certain embodiments of the present disclosure.

FIG. 4 shows a schematic of sensor assembly circuitry, in accordance with certain embodiments of the present disclosure.

FIG. 5 shows a schematic of sensor circuitry, in accordance with certain embodiments of the present disclosure.

FIG. 6 shows a schematic of sensor assembly circuitry, in accordance with certain embodiments of the present disclosure.

FIG. 7 shows a schematic of sensor circuitry, in accordance with certain embodiments of the present disclosure.

FIG. 8 shows a schematic of sensor assembly circuitry, in accordance with certain embodiments of the present disclosure.

While the disclosure is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the invention as defined by the appended claims.

### DETAILED DESCRIPTION

During medical procedures, medical devices such as probes (e.g., catheters) are inserted into a patient through the patient's vascular system and/or a catheter lumen. To track the location and orientation of a probe within the patient, probes can be provisioned with magnetic field sensors.

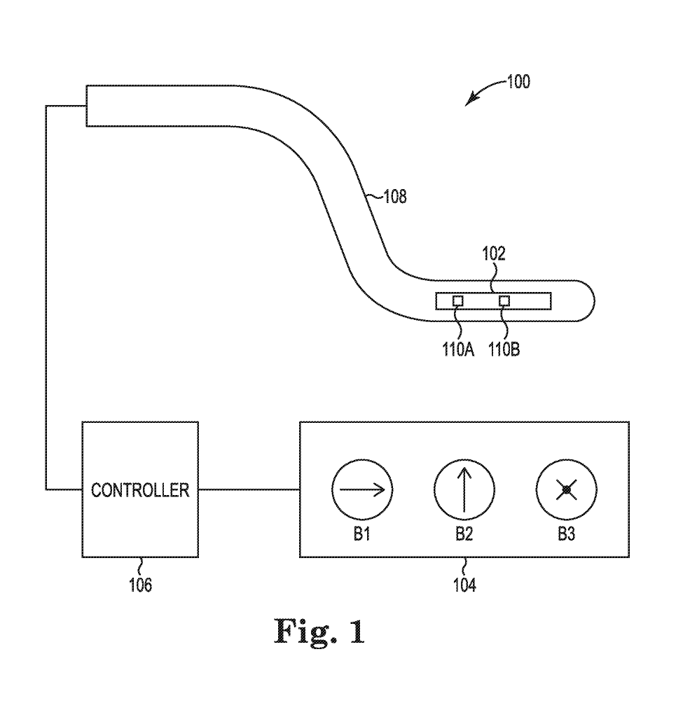

FIG. 1 is a diagram illustrating a tracking system 100 including a sensor assembly 102, a magnetic field generator 104, a controller 106, and a probe 108 (e.g., catheter, imaging probe, diagnostic probe). As shown, the sensor assembly 102 can be positioned within the probe 108, for example, at a distal end of the probe 108. The tracking system 100 is configured to determine the location and orientation of the sensor assembly 102 and, therefore, the probe 108. Magnetic fields generated by the magnetic field generator 104 provide a frame of reference for the tracking system 100 such that the location and orientation of the sensor assembly 102 within the generated magnetic fields can be determined. The tracking system 100 can be used in a medical procedure, where the probe 108 is inserted into a patient and the sensor assembly 102 is used to assist with tracking the location of the probe 108 in the patient.

In various embodiments, the probe 108 may include, for example, a catheter (e.g., a mapping catheter, an ablation catheter, a diagnostic catheter, introducer, etc.), an endoscopic probe or cannula, an implantable medical device (e.g., a control device, a monitoring device, a pacemaker, an implantable cardioverter defibrillator (ICD), a cardiac resynchronization therapy (CRT) device, a CRT-D device, etc.), and/or the like. For example, in embodiments, the probe 108 may include a mapping catheter associated with an anatomical mapping system. The probe 108 may include any other type of device configured to be at least temporarily disposed within a subject.

The sensor assembly 102 is communicatively coupled to the controller 106 by a wired or wireless communications path such that the controller 106 sends and receives various signals to and from the sensor assembly 102. The magnetic field generator 104 is configured to generate one or more magnetic fields. For example, the magnetic field generator 104 is configured to generate at least three magnetic fields B1, B2, and B3, each generated by a respective magnetic field transmitter (e.g., a coil). The controller 106 is configured to control the magnetic field generator 104 via a wired or wireless communications path to generate one or more of the magnetic fields B1, B2, and B3 to assist with tracking the sensor assembly 102 (and therefore probe 108).

In various embodiments, the controller 106 includes a signal generator configured to provide driving current to each of the magnetic field transmitters, causing each magnetic field transmitter assembly to transmit an electromagnetic field. In certain embodiments, the controller 106 is configured to provide variable (e.g., sinusoidal) driving currents to the magnetic field transmitters within the magnetic field generator 104. The controller 106 can be implemented using firmware, integrated circuits, and/or software modules that interact with each other or are combined together. For example, the controller 106 may include computer-readable instructions/code for execution by a processor within or associated with the controller 106. Such instructions may be stored on a non-transitory computer-readable medium and transferred to the processor for execution. In some embodiments, the controller 106 can be implemented in one or more application-specific integrated circuits and/or other forms of circuitry suitable for controlling and processing magnetic tracking signals and information.

The sensor assembly 102 is configured to sense the generated magnetic fields and provide tracking signals indicating the location and orientation of the sensor assembly 102 in up to six degrees of freedom (i.e., x, y, and z measurements, and pitch, yaw, and roll angles). Generally, the number of degrees of freedom that a tracking system is able to track depends on the number of magnetic field sensors and magnetic field generators. For example, a tracking system with a single magnetic field sensor may not be capable of tracking roll angles and thus are limited to tracking in only five degrees of freedom (i.e., x, y, and z coordinates, and pitch and yaw angles). This is because a magnetic field sensed by a single magnetic field sensor does not change as the single magnetic field sensor is “rolled.” As such, the sensor assembly 102 includes at least two magnetic field sensors, 110A and 110B. The magnetic field sensors can include sensors such as inductive sensing coils and/or various sensing elements such as magneto-resistive (MR) sensing elements (e.g., anisotropic magneto-resistive (AMR) sensing elements, giant magneto-resistive (GMR) sensing elements, tunneling magneto-resistive (TMR) sensing elements, Hall effect sensing elements, colossal magneto-resistive (CMR) sensing elements, extraordinary magneto-resistive (EMR) sensing elements, spin Hall sensing elements, and the like), giant magneto-impedance (GMI) sensing elements, and/or flux-gate sensing elements. In addition, the sensor assembly 102 and/or the probe 108 can feature other types of sensors, such as temperature sensors, ultrasound sensors, etc.

The sensor assembly 102 is configured to sense each of the magnetic fields B1, B2, and B3 and provide signals to the controller 106 that correspond to each of the sensed magnetic fields B1, B2, and B3. The controller 106 receives the signals from the sensor assembly 102 via the communications path and determines the position and location of the sensor assembly 102 and probe 108 in relation to the generated magnetic fields B1, B2, and B3.

The magnetic field sensors can be powered by voltages or currents to drive or excite elements of the magnetic field sensors. The magnetic field sensor elements receive the voltage or current and, in response to one or more of the generated magnetic fields, the magnetic field sensor elements generate sensing signals, which are transmitted to the controller 106. The controller 106 is configured to control the amount of voltage or current to the magnetic field sensors and to control the magnetic field generators 104 to generate one or more of the magnetic fields B1, B2, and B3. The controller 106 is further configured to receive the sensing signals from the magnetic field sensors and to determine the location and orientation of the sensor assembly 102 (and therefore probe 108) in relation to the magnetic fields B1, B2, and B3. The controller 106 can be implemented using firmware, integrated circuits, and/or software modules that interact with each other or are combined together. For example, the controller 106 may include computer-readable instructions/code for execution by a processor. Such instructions may be stored on a non-transitory computer-readable medium and transferred to the processor for execution. In general, the controller 106 can be implemented in any form of circuitry suitable for controlling and processing magnetic tracking signals and information.

In the illustrated embodiment the controller 106 is shown as a single functional block that controls the operation of the magnetic field generator 104 and also receives and processes the signals from the sensor assembly 102 corresponding to the sensed magnetic fields B1, B2, B3 for tracking the position and orientation of the probe 108 within the multi-dimensional magnetic field generated by the magnetic field generator 104. The skilled artisan will appreciate that the foregoing functionality may be implemented in one or more hardware and software components/systems. For example, in embodiments, the controller 106 functionality relating to control of the magnetic field generator 104 and the processing of the signals from the sensor assembly 102 may be performed by a single processor. In other embodiments, these functions may be performed in multiple processors.

In various embodiments, the magnetic field sensors 110a, 110b are disposed on a substrate as part of the sensor assembly 102. In embodiments, the substrate may be a flexible substrate. In embodiments, the magnetic field sensors 110a, 110b may be oriented so as to be sensitive to components of the generated magnetic field in different directions. In embodiments, the directions of sensitivity may be orthogonal to one another. In various embodiments, the magnetic field sensors 110a, 110b may lie in the same plane, but be oriented in different directions. In other embodiments, the substrate may include a first portion oriented in a first plane, with the magnetic field sensor 110a being located thereon, and may also include a second portion oriented in a second plane with the magnetic field sensor 110b located thereon. In embodiments, the first and second planes may be orthogonal to one another.

Although in the illustrated embodiment the sensor assembly 102 includes two magnetic field sensors 110a, 110b, in other embodiments the sensor assembly 102 may include additional magnetic field sensors.

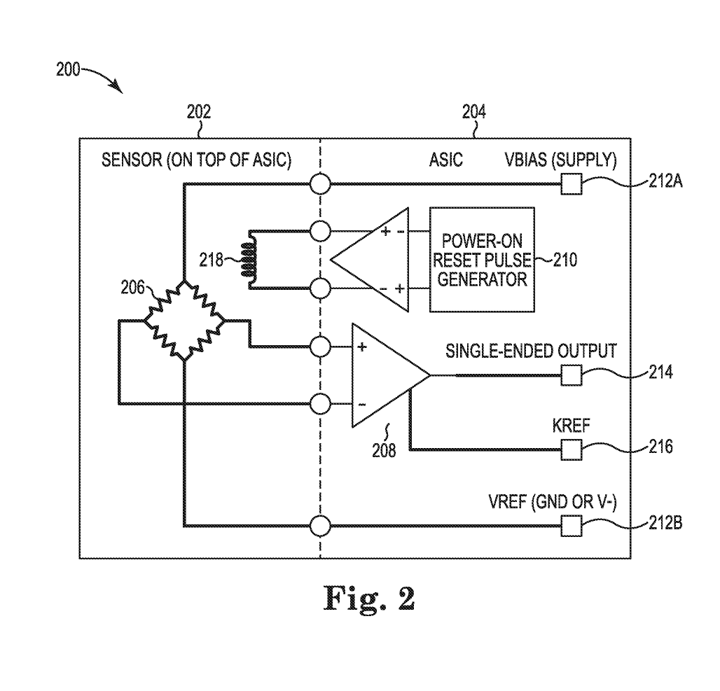

FIG. 2 shows sensor circuitry 200 for a magnetic field sensor such as the magnetic field sensor 110A or 110B of FIG. 1. The sensor circuitry 200 includes a sensor portion 202 and an application-specific integrated circuit (ASIC) portion 204. As shown in FIG. 2, the sensor portion 202 and the ASIC portion 204 can be implemented on the same die or substrate (e.g., a monolithic design). For example, the sensor portion 202 can be fabricated on top of the ASIC portion 204. In some embodiments, the sensor portion 202 and the ASIC portion 204 can be implemented on separate dies and positioned next to each other. In such embodiments, the sensor portion 202 and the ASIC portion 204 can be electrically and communicatively coupled together.

The sensor portion 202 includes one or more MR sensing elements 206, which can be AMR sensing elements, GMR sensing elements, TMR sensing elements, CMR sensing elements, EMR sensing elements, and the like. The MR sensing elements 206 are configured to sense magnetic fields, like those generated by the magnetic field generator 104 of FIG. 1, and generate a sensing signal. In some embodiments, the MR sensing elements 206 can be arranged in a Wheatstone bridge configuration as shown in FIG. 2, where four MR sensing elements are connected together to make a bridge circuit. In such embodiments, a change in one or more of the MR sensing elements in the bridge circuit, due to the sensed magnetic field, will result in a differential voltage output from the bridge circuit, so as to generate the sensing signal. In some embodiments, a single MR sensing element can be used to sense magnetic fields.

The ASIC portion 204 includes various integrated circuits such as an amplifier circuit 208 and a reset circuit 210, which can be fashioned using any suitable semiconductor technology. The ASIC portion 204 also includes bias connections, 212A and 212B, which are used to provide a bias current to the MR sensing elements 206 from a supply source (not shown), and also to provide power to the ASIC portion 204.

The amplifier circuit 208 operates to increase the signal strength of the generated responsive sensing signal from the MR sensing elements 206. Accordingly, the amplifier circuit 208 includes an output connection 214 and a Kelvin connection 216. The Kelvin connection 216 is operable to compensate for voltage losses caused by line resistances, which would otherwise cause errors in low voltage measurements, and to define the reference voltage for the amplifier circuit 208 output (i.e., when the input signal to the amplifier circuit 208 is zero, the output from the amplifier circuit 208 is equal to the reference voltage).

The reset circuit 210 operates to reset the one or more MR sensing elements 206. Accordingly, the reset circuit 210 includes a reset coil 218 constructed near the MR sensing elements 206 on the sensor portion 202. After exposure to external magnetic fields such as the magnetic fields B1, B2, and B3 of FIG. 1, the MR sensing elements 206 typically require the application of a magnetic field to reset their magnetic sensitivities. That is, by resetting the magneto-resistive film domains in the MR sensing elements 206 to a previous or relatively-known magnetic orientation. This is accomplished when the reset circuit 210 generates a current pulse through the reset coil 218 to create the magnetic field needed for the reset. For example, the reset circuit 210 can generate the current pulse at the system power-on stage to reset the MR sensing elements 206.

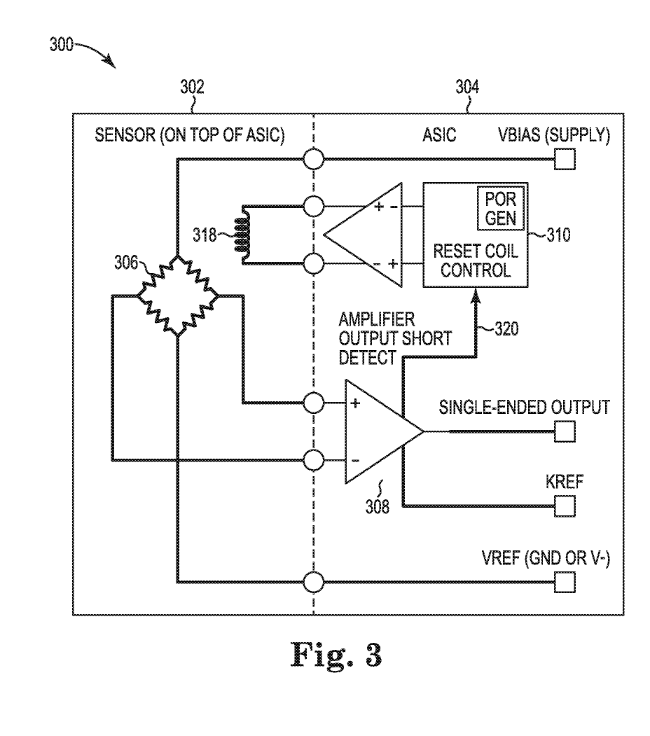

FIG. 3 shows a sensor circuitry 300 for reset control of a magnetic field sensor such as the magnetic field sensor 110A or 110B of FIG. 1. The sensor circuitry 300 is similar to the sensor circuitry 200 and includes a sensor portion 302 and an ASIC portion 304. The sensor portion 302 includes MR sensing elements 306 and a reset coil 318. The ASIC portion 304 includes various integrated circuits such as an amplifier circuit 308 and a reset circuit 310 that controls the reset coil 318. In embodiments where the sensor portion 302 and ASIC portion 304 are configured on separate dies, the reset coil 318 can be part of the sensor portion 302.

The sensor circuitry 300 uses the output of the amplifier circuit 308 to activate the reset coil 318. In particular, upon detecting a triggering event, a reset control signal 320 is generated and sent to the reset circuit 310. In one embodiment, the triggering event can include the amplifier output being short circuited (e.g., to the supply source or ground) by control circuitry of the controller 106 (see FIG. 1). In response to the receiving the reset control signal 320, the reset circuit 310 generates a current pulse through the reset coil 318 to create a magnetic field that will reset the MR sensing elements 306. In some embodiments, the output of the amplifier circuit 308 is used as the control signal 320. This approach has the advantage of generating an on-chip control signal for the reset rather than having a separate and extra control signal line to perform the reset—reducing the number of conductors (e.g., wires) coupled to the sensor circuitry 300. The pulse time for the generated current pulse can be predetermined. In some embodiments, the pulse time is based on the length of time that the output of the amplifier circuit 308 is shorted.

The output of the amplifier circuit 308 can be shorted by, for example, a controller such as the controller 106 of FIG. 1. In some embodiments, the output can be shorted automatically at the system power-on stage. In some embodiments, the output can be shorted manually at any time. In other embodiments, both the amplifier output detection reset and the power-on reset can be implemented in the reset circuit 310.

Further, as shown in FIG. 3, two drive signals from the reset circuit 310 are used to activate the reset coil 318. However, in some embodiments, one side of the reset coil 318 can be connected to either the supply source or ground. In this manner, only a single drive signal is needed to activate the reset coil 318.

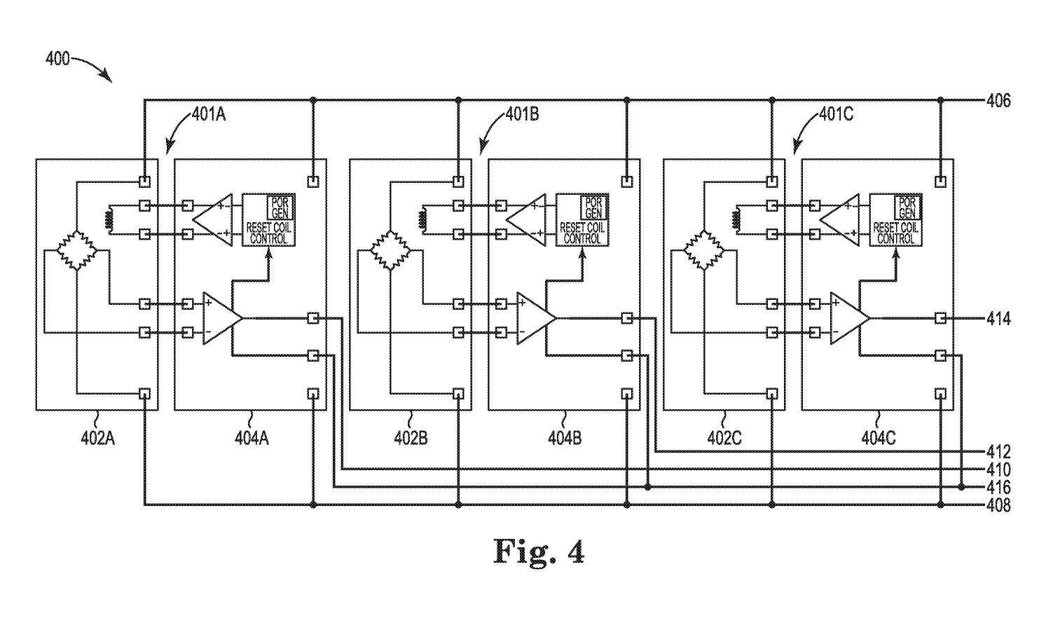

FIG. 4 shows a sensor assembly circuitry 400 for reset control of a sensor assembly used in tracking systems such as the sensor assembly 102 used in the tracking system 100 of FIG. 1. The sensor assembly circuitry 400 is comprised of a first sensor circuitry 401A for a first magnetic field sensor, a second sensor circuitry 401B for a second magnetic field sensor, and a third sensor circuitry 401C for a third magnetic field sensor. The first sensor circuitry 401A includes a separate sensor portion 402A and a separate ASIC portion 404A. Similarly, the second sensor circuitry 401B includes a separate sensor portion 402B and a separate ASIC portion 404B, while the third sensor circuitry 401C includes a separate sensor portion 402C and a separate ASIC portion 404C. However, each of the sensor circuitries 401A-C can also be implemented monolithically like the sensor circuitry 300 of FIG. 3. As shown in FIG. 4, the sensor assembly circuitry 400 has six signal lines: supply source bias (406), ground (408), generated sensing signals (410-414), and Kelvin connection (416).

Similar to the sensor circuitry 300 of FIG. 3, reset control can be accomplished by using amplifier output detection in each of the sensor circuitries 401A-C. Moreover, each magnetic field sensor can be reset one at a time to reduce the amount of current sent to the circuitry at the same time.

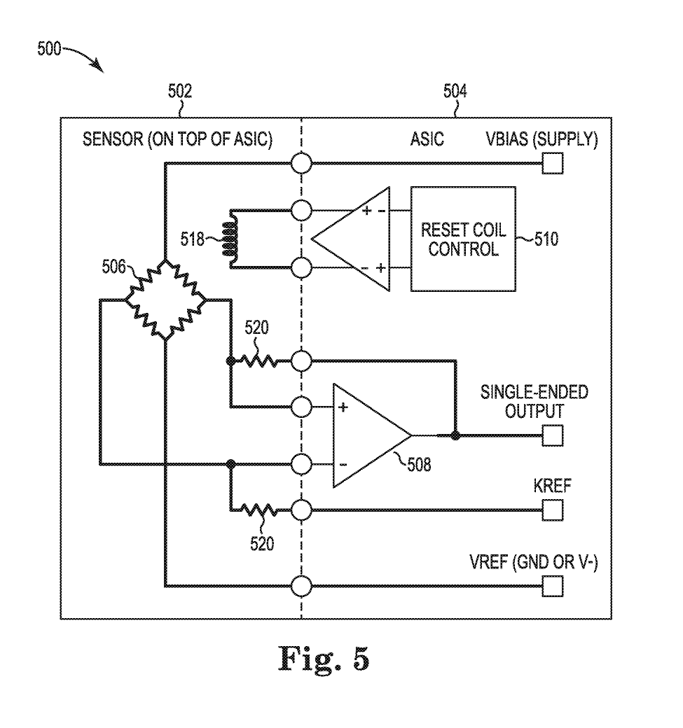

FIG. 5 shows sensor circuitry 500 for gain setting of a magnetic field sensor such as the magnetic field sensor 110A or 110B of FIG. 1. The sensor circuitry 500 is similar to the sensor circuitry 200 and includes a sensor portion 502 and an ASIC portion 504. The sensor portion 502 includes MR sensing elements 506, a reset coil 518, and gain setting resistors 520. The ASIC portion 504 includes various integrated circuits such as an amplifier circuit 508 and a reset circuit 510 that controls the reset coil 518. In various embodiments, the reset circuit 510 can be configured in substantially the same manner as the reset circuit 310 described in connection with the embodiment of FIGS. 3-4.

The sensor circuitry 500 uses feedback resistance from the gain setting resistors 520 to match variations in the MR sensing elements 506. In particular, a gain is determined by the ratio of the feedback resistance from the gain setting resistors 520 to the resistance of the MR sensing elements 506. The gain can be used to cancel out any variations (e.g., production) in the resistance of the MR sensing elements 506. This approach has the advantage of enabling the use of a single ASIC design with different sensor designs having, for example, different sensitivities. For example, as the gain setting resistors 520 are constructed on the sensor portion 502, a manufacturer can engineer the output of the MR sensing elements 506 (by tuning the values of the gain setting resistors 520) to meet the input requirements of the ASIC portion 504. The values of the gain setting resistors 520 can be selected based on the MR sensing elements 506. In some embodiments, resistor trimming can be used to adjust the values of the gain setting resistors 520.

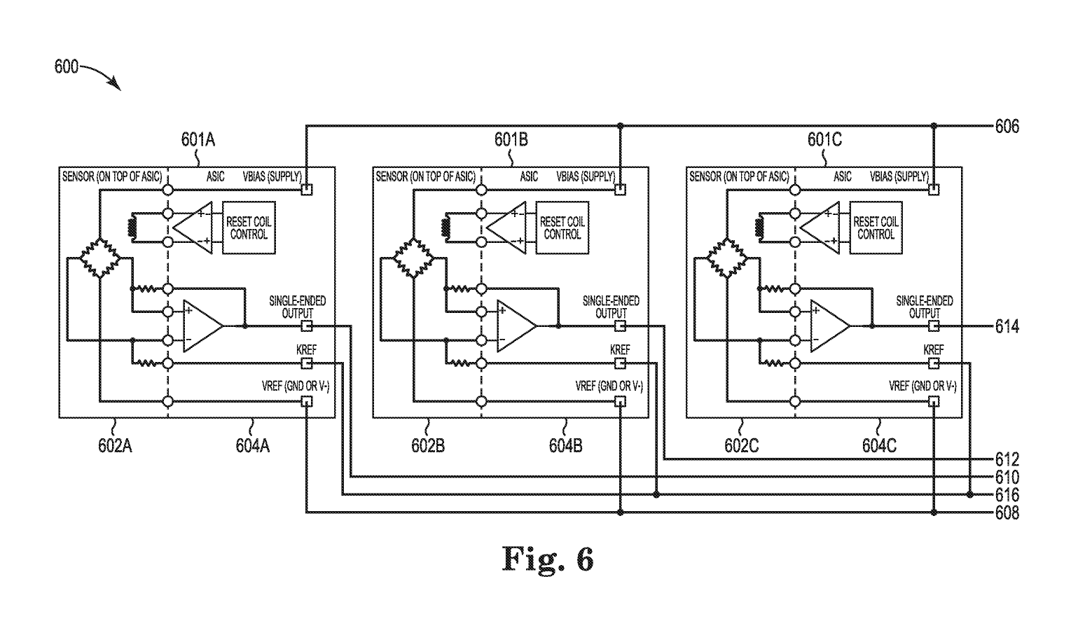

FIG. 6 shows sensor assembly circuitry 600 for gain setting of a sensor assembly used in tracking systems such as the sensor assembly 102 used in the tracking system 100 of FIG. 1. The sensor assembly circuitry 600 is comprised of a first sensor circuitry 601A for a first magnetic field sensor, a second sensor circuitry 601B for a second magnetic field sensor, and a third sensor circuitry 601C for a third magnetic field sensor. Each of the sensor circuitries 601A-C is similar to the sensor circuitry 500 of FIG. 5 (i.e., the first sensor circuitry 601A includes a monolithic sensor portion 602A and ASIC portion 604A, the second sensor circuitry 601B includes a monolithic sensor portion 602B and ASIC portion 604B, and the third sensor circuitry 601C includes a monolithic sensor portion 602C and ASIC portion 604C). As shown in FIG. 6, the sensor assembly circuitry 600 has six signal lines: supply source bias (606), ground (608), generated sensing signals (610-614), and Kelvin connection (616).

Similar to the sensor circuitry 500 of FIG. 5, each of the sensor circuitries 601A-C can use feedback resistance from gain setting resistors to match variations in the magnetic field sensors. In this manner, the ASIC design will not require modifications for different sensor designs, as the gain setting resistors can be modified to compensate for changes in the sensors.

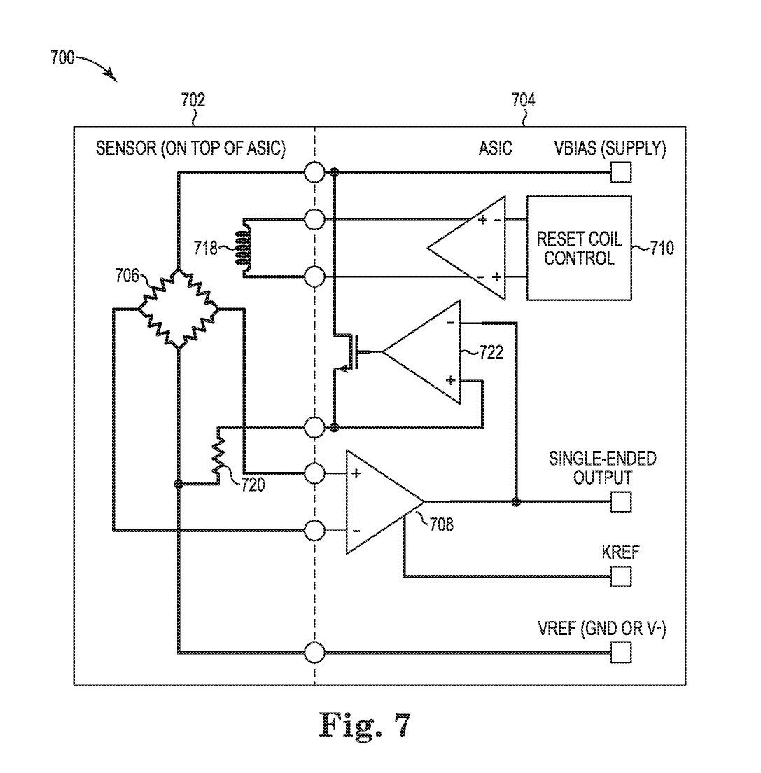

FIG. 7 shows sensor circuitry 700 for bias current compensation of a magnetic field sensor such as the magnetic field sensor 110A or 110B of FIG. 1. The sensor circuitry 700 is similar to the sensor circuitry 200 and includes a sensor portion 702 and an ASIC portion 704. The sensor portion 702 includes MR sensing elements 706, a reset coil 718, and a tuning resistor 720. The ASIC portion 704 includes various integrated circuits such as an amplifier circuit 708, a reset circuit 710 that controls the reset coil 718, and a bias current (or bias signal) compensation circuit 722. In various embodiments, the reset circuit 710 can be configured in substantially the same manner as the reset circuit 310 described in connection with the embodiment of FIGS. 3-4.

The sensor circuitry 700 generates a compensation current or signal to compensate for variations in the bias current of the MR sensing elements 706. The bias current through the MR sensing elements 706 can vary as the sensed magnetic field varies. Accordingly, if the bias current compensation circuit 722 detects that the bias current through the MR sensing elements 706 is decreasing, then the bias current compensation circuit 722 will increase the compensation current. On the other hand, if the bias current compensation circuit 722 detects that the bias current through the MR sensing elements 706 is increasing, then the bias current compensation circuit 722 will decrease the compensation current. This approach has the advantage of producing a net DC bias current for the sensor and ASIC combination. The tuning resistor 720 can be used to set the gain (transconductance) of the compensation current. The value of tuning resistor 720 can be selected based on the MR sensing elements 706.

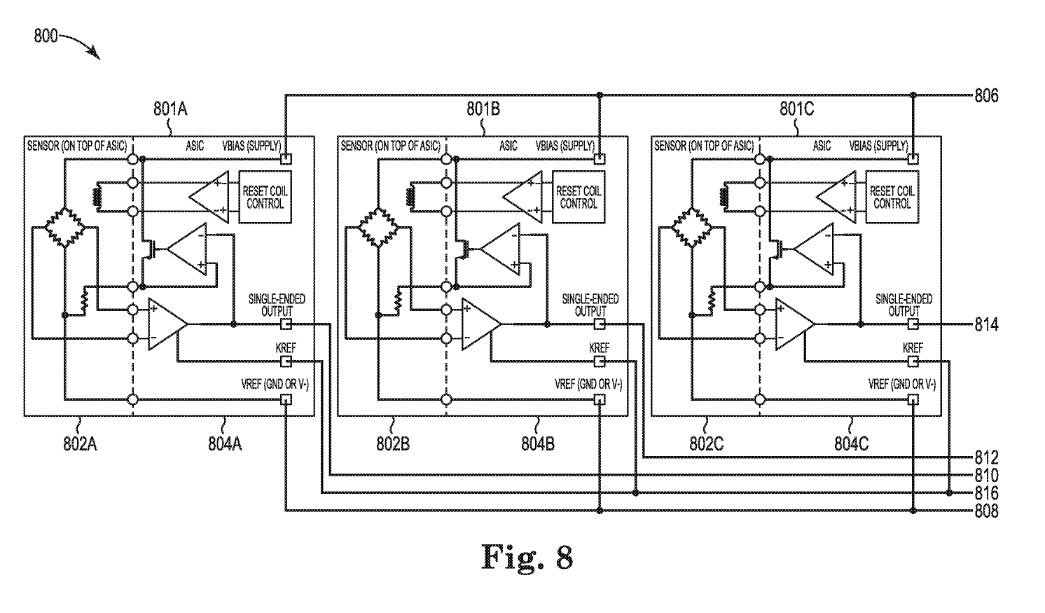

FIG. 8 shows sensor assembly circuitry 800 for bias current compensation of a sensor assembly used in tracking systems such as the sensor assembly 102 used in the tracking system 100 of FIG. 1. The sensor assembly circuitry 800 is comprised of a first sensor circuitry 801A for a first magnetic field sensor, a second sensor circuitry 801B for a second magnetic field sensor, and a third sensor circuitry 801C for a third magnetic field sensor. Each of the sensor circuitries 801A-C is similar to the sensor circuitry 700 of FIG. 7 (i.e., the first sensor circuitry 801A includes a monolithic sensor portion 802A and ASIC portion 804A, the second sensor circuitry 801B includes a monolithic sensor portion 802B and ASIC portion 804B, and the third sensor circuitry 801C includes a monolithic sensor portion 802C and ASIC portion 804C). As shown in FIG. 8, the sensor assembly circuitry 800 has six signal lines: supply source bias (806), ground (808), generated sensing signals (810-814), and Kelvin connection (816).

Similar to the sensor circuitry 700 of FIG. 7, each of the sensor circuitries 801A-C can generate a compensation current or signal to compensate for variations in the bias current of the magnetic field sensors. For example, in one embodiment, magnetic censor circuitry 801C may be located distally of the sensor circuitry 801A and 801B along a common substrate, and consequently, the bias current for the sensor circuitry 801C will pass by the sensor circuitry 801A and 801B in operation. Because the signal at each sensor varies, the bias current for each sensor may also vary, which can generate a small magnetic field that could be sensed by the other magnetic sensors. To avoid this crosstalk, the bias current compensation circuit for the sensor circuitry 801C can generate a current that is equal to but opposite of the output current signal generated by that magnetic sensor, such that the net current passing by the other magnetic sensors does not vary. In this manner, a constant net current can be provided to a single bias line for the three sensor and ASIC combinations.

The embodiments shown in FIGS. 3, 5, and 7 are not mutually exclusive and can be used in combination with each other. Further, while FIGS. 4, 6, and 8 are shown as having three magnetic field sensors, it is appreciated that there could be only two magnetic field sensors or more than three magnetic field sensors. Moreover, while FIGS. 4, 6, and 8 show that each magnetic field sensor is supported by an ASIC, in some embodiments, a single ASIC could support multiple magnetic field sensors.

The present disclosure provides the advantages of minimizing the number of signal lines when using MR technology. For example, the number of signal lines is reduced by using current mode signaling and by using the amplifier output for reset control. Moreover, by compensating for sensor bias current variations, the amount of magnetic coupling (crosstalk) from a distal sensor to a proximal sensor can be reduced.

It should be noted that, for simplicity and ease of understanding, the elements described above and shown in the figures are not drawn to scale and may omit certain features. As such, the drawings do not necessarily indicate the relative sizes of the elements or the non-existence of other features.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

We claim:

1. A sensing apparatus comprising:
a magneto-resistive (MR) sensing element for sensing magnetic fields;
a reset coil element configured to generate a reset field for resetting a magnetic orientation of the MR sensing element; and
semiconductor circuitry including:
a reset circuit configured to supply a reset current to the reset coil element, and
an amplifier circuit coupled to the MR sensing element, the amplifier circuit including an amplifier output and configured to generate a sensing signal proportional to the sensed magnetic fields,
wherein the MR sensing element and the coil element are formed on a first chip and the semiconductor circuitry is formed on a second chip.

2. The sensing apparatus of claim 1, wherein the semiconductor circuitry is further configured to generate a reset control signal operative to activate the reset circuit and cause the reset circuit to supply the reset current to the reset coil element.

3. The sensing apparatus of claim 2, wherein the amplifier circuit is configured to generate the reset control signal upon detecting a triggering event.

4. The sensing apparatus of claim 3, wherein the triggering event includes the amplifier output being short-circuited.

5. The sensing apparatus of claim 4, further comprising:
a gain setting resistor, wherein the amplifier circuit is further coupled to the gain setting resistor; and
wherein the sensing signal is based on a gain determined from a ratio of a resistance value of gain setting resistor to a resistance value of the MR sensing element.

6. The sensing apparatus of claim 5, wherein the gain setting resistor is formed on the first chip.

7. The sensing apparatus of claim 6, wherein the semiconductor circuitry further includes:
a bias signal compensation circuit, wherein the amplifier circuit is further coupled to the bias signal compensation circuit; and
wherein the bias signal compensation circuit generates a compensation signal based on the sensing signal, the compensation signal to be combined with a bias signal to the MR sensing element.

8. The sensing apparatus of claim 7, further comprising a tuning resistor formed on the first chip, wherein the compensation signal is based on a resistance value of the tuning resistor.

9. The sensing apparatus of claim 7, wherein the bias signal compensation circuit increases the compensation signal when the bias signal to the MR sensing element decreases, and decreases the compensation signal when the bias signal to the MR sensing element increases.

10. The sensing apparatus of claim 7, wherein the first chip is formed on top of the second chip.

11. A sensing apparatus comprising:
a magneto-resistive (MR) sensing element for sensing magnetic fields;
semiconductor circuitry including an amplifier circuit coupled to the MR sensing element, the amplifier circuit including an amplifier output and configured to generate a sensing signal proportional to the sensed magnetic fields; and
a gain setting resistor coupled to the amplifier circuit, wherein the sensing signal is based on a gain determined from a ratio of a resistance value of the gain setting resistor to a resistance value of the MR sensing element, and
wherein the MR sensing element is formed on a first chip and the semiconductor circuitry is formed on a second chip.

12. The sensing apparatus of claim 11, wherein the gain setting resistor is formed on the first chip.

13. The sensing apparatus of claim 11, wherein the first chip is formed on top of the second chip.

14. The sensing apparatus of claim 11, wherein the semiconductor circuitry further includes:
a bias signal compensation circuit, wherein the amplifier circuit is further coupled to the bias signal compensation circuit; and
wherein the bias signal compensation circuit generates a compensation signal based on the sensing signal, the compensation signal to be combined with a bias signal to the MR sensing element.

15. The sensing apparatus of claim 14, further comprising a tuning resistor formed on the first chip, wherein the compensation signal is based on a resistance value of the tuning resistor.

16. The sensing apparatus of claim 14, wherein the bias signal compensation circuit increases the compensation signal when the bias signal to the MR sensing element decreases, and decreases the compensation signal when the bias signal to the MR sensing element increases.

17. A sensing apparatus comprising:
a magneto-resistive (MR) sensing element for sensing magnetic fields; and
semiconductor circuitry including:
an amplifier circuit coupled to the MR sensing element, the amplifier circuit including an amplifier output and configured to generate a sensing signal proportional to the sensed magnetic fields; and
a bias signal compensation circuit coupled to the amplifier circuit, wherein the bias signal compensation circuit generates a compensation signal based on the sensing signal, the compensation signal to be combined with a bias signal to the MR sensing element, and
wherein the MR sensing element is formed on a first chip and the semiconductor circuitry is formed on a second chip.

18. The sensing apparatus of claim 17, further comprising a tuning resistor formed on the first chip, wherein the compensation signal is based on a resistance value of the tuning resistor.

19. The sensing apparatus of claim 17, wherein the bias signal compensation circuit increases the compensation signal when the bias signal to the MR sensing element decreases, and decreases the compensation signal when the bias signal to the MR sensing element increases.

20. The sensing apparatus of claim 17, wherein the first chip is formed on top of the second chip.
